(19) United States
(12) Patent Application Publication
(10) Pub. No.: US 2017/0151367 A1
(43) Pub. Date: Jun. 1, 2017

(54) DRY DERMAL FILLER COMPOSITIONS AND METHODS OF RECONSTITUTION

(71) Applicant: Allergan, Inc., Irvine, CA (US)

(72) Inventors: Xiaojie Yu, Irvine, CA (US); Jacob F. Pollock, Honolulu, HI (US); Nicholas Manesis, Escondido, CA (US)

(21) Appl. No.: 15/428,985

(22) Filed: Feb. 9, 2017

Related U.S. Application Data
(62) Division of application No. 14/693,628, filed on Apr. 22, 2015.
(60) Provisional application No. 61/982,817, filed on Apr. 22, 2014.

(51) Int. Cl. A61L 27/20; A61L 27/54; A61L 27/52; A61L 27/50

(52) U.S. Cl. CPC A61L 27/20; A61L 27/50; A61L 27/54; A61L 27/52; A61L 2400/06; A61L 2300/402; A61L 2300/43; A61L 2300/45; A61L 2430/34

(57) ABSTRACT

Dermal fillers are provided, particularly hyaluronic acid-based dermal fillers containing additives, for example, unstable additives, the dermal fillers being provided in an anhydrous state for extended shelf life.

This application is a divisional of U.S. patent application Ser. No. 14/693,628, filed on Apr. 22, 2015, which claims priority to, and the benefit of, U.S. Provisional Patent Application No. 61/982,817, filed on Apr. 22, 2014, the entire specification of which is incorporated herein by this reference.

The present invention generally relates to dermal fillers and more specifically relates to dry dermal filler compositions including additives.

### SUMMARY

In accordance with an embodiment of the invention, dermal fillers with additives, for example, unstable additives and/or sustained release additives, are lyophilized to the dry state for extended shelf life. The dry fillers are reconstituted in the physicians' clinics prior to injection using water for injection or phosphate buffered saline with or without additional additives such as soluble hyaluronic acid.

In one embodiment, a dermal filler material is provided which can be reconstituted and injected into skin. In one embodiment, the filler comprises a crosslinked polysaccharide, for example, crosslinked hyaluronic acid (HA) and a beneficial additive. The additive may be epinephrine, phenylephrine, or another beneficial agent that is relatively stable in the dry state but decomposes in the wet state.

The present invention also provides methods of making a dry dermal filler for reconstitution, as well as methods for treating skin using reconstituted dermal filler.

The present invention may be more clearly understood with respect to the following Detailed Description and accompanying Drawings which show:

### BRIEF DESCRIPTION OF THE DRAWINGS

FIG. 1 shows epinephrine degradation to adrenochrome over 48 hrs in HA gel in wet form.

FIG. 2 shows epinephrine and adrenochrome concentrations in gels stored in wet and dry form after 48 hrs.

FIG. 3 shows epinephrine concentration in gels stored in wet and dry form during accelerated aging at 50° C.

### DETAILED DESCRIPTION

In one embodiment, a dry dermal filler composition in powdered form is provided. The composition may be made from a hyaluronic-acid based dermal filler gel, which has been subjected to processes to cause the gel to become anhydrous. For example, the gel is lyophilized using freeze drying techniques, for example.

The composition may comprise a glycosaminoglycan polymer hydrogel. The glycosaminoglycan may be a hyaluronic acid, for example, sodium hyaluronate. The hyaluronic acid may comprise crosslinked hyaluronic acid. Suitable crosslinking agents are well known and include dialdehydes and disulfides crosslinking agents including, without limitation, divinyl sulfones, diglycidyl ethers, and bis-epoxides, and biscarbodiimide. Non-limiting examples of hyaluronan crosslinking agents include divinyl sulfone (DVS), 1,4-butanediol diglycidyl ether (BDDE), and others known to those of skill in the art.

In some embodiments, the composition contains an additive. In one embodiment, the additive is one that is generally unstable when in solution, but is relatively stable when in a dry state. For example, the additive may comprise epinephrine, phenylephrine, and optionally one or more other additives, such as lidocaine. In some embodiments, the additive comprises naphazoline, epinephrine, methoxamine, methylnorepinephrine, norepinephrine, oxymetazoline, phenylephrine, pseudoephedrine, synephrine, cirazoline, xylometazoline, an analog or a derivative thereof, or any combination thereof. In some embodiments, the additive is an anesthetic agent, for example, lidocaine, or a similar agent.

The lyophilized composition (hereinafter sometimes referred to as a “dry dermal filler composition”, “dry composition”, or “powder composition”), is contained in a syringe. The dry composition may be maintained under vacuum in the syringe. Advantageously, the dry composition has an increased shelf life, for example, relative to the gel precursor from which it was made. Also advantageously, many of the dry compositions of the invention, when reconstituted with a liquid such as water, for example, have similar or comparable rheological properties to the gel precursor from which they are made.

For use as a dermal filler, the dry composition may be contacted and combined with a liquid component, for example, saline or water. The composition is allowed to hydrate until the composition forms a hydrated gel which is suitable as a dermal filler for dermal or subcutaneous injection. The dermal filler is useful for filling in wrinkles, reducing the appearance of fine lines and/or adding volume or contour to the face.

Rehydration of the dry composition may be accomplished, for example, by introducing water or other solution, through a septum located on the distal end of the syringe containing the dry composition. The septum may be pierced, for example, using a cannula or needle to allow the introduction of a suitable amount of the reconstituting liquid into the syringe barrel containing the powder composition. Once contacted with the water or other solution, the powder composition become hydrated and forms a viscous gel suitable for injection into skin. For example, the septum may then be removed from the syringe, and the syringe may be coupled to a needle in the usual fashion to enable injection of the reconstituted gel into skin, for example, into the dermis or subcutaneous region, for example, in a manner as is normally done with a conventional dermal filler.

In another embodiment, the dry composition in the syringe is not stored under vacuum. In another embodiment, water for reconstitution may be added through a septum on the plunger end of the syringe rather than the distal end of the syringe. In yet another embodiment, a dermal filler with additives is lyophilized in one chamber of a dual chamber syringe. The liquid component, for example, water, is stored in the other chamber until reconstitution is desired. The dual chamber syringe may be designed to allow aseptic addition of liquid to the dry composition, for example, through a by-pass channel.

In some embodiments, the dry composition can be a hyaluronic acid based gel including no additives, which is mixed in the dry state with a dry additive, and the dry mixture loaded into a syringe for reconstitution.

Accordingly, in one aspect, the present invention provide a material suitable for reconstitution and injection into skin as a dermal filler, the material made by the process of providing a gel comprising crosslinked hyaluronic acid and at least one additive, lyophilizing the gel to obtain a dry gel suitable for reconstitution; wherein the additive remains stable in the dry gel. The additive may be lidocaine, epinephrine, phenylephrine, or any combination thereof. The additive may comprise, for example, any additive that is beneficial to the patient, for example, to reduce pain or bruising upon injection, and which is stable when present in a dry form,

In another aspect, a method of making a material suitable for reconstitution and injection into skin as a dermal filler is provided, the method comprising providing a gel comprising crosslinked hyaluronic acid and at least one additive; lyophilizing the gel to obtain a dry gel suitable for reconstitution; wherein the additive remains stable in the dry gel. The additive may be lidocaine, epinephrine, phenylephrine, or any combination thereof.

In yet another aspect, a method of making a dermal filler is provided, the method comprising providing a dry gel comprising crosslinked hyaluronic acid and at least one additive, and reconstituting the dry gel with an aqueous solution prior to injection into the skin.

In another aspect, a method of making a dermal filler is provided, comprising providing a dry gel as described elsewhere herein, for example a lyophilized gel comprising crosslinked hyaluronic acid and at least one additive, and reconstituting the dry gel. The step of reconstituting may comprise adding an aqueous solution, for example, water, saline or another suitable aqueous solution, to the dry gel to create an injectable, hydrated gel suitable for injection into skin as a dermal filler. The step of reconstituting may comprises adding the water or saline to the dry gel, for example, while the dry gel is contained in a syringe, and using the same syringe for injection into a patient. In some embodiments, the step of reconstituting comprises adding soluble hyaluronic acid, for example, in solution, to the dry gel.

### EXAMPLE 1

20 syringes of Juvederm® Ultra Plus dermal filler (a commercial hyaluronic acid-based injectable gel, containing BDDE-crosslinked hyaluronic acid, with a HA concentration of 24 mg/g, manufactured by Allergan, Inc.) were pooled to give 20 mL of gel. The gel was the mixed with 60 mg of dry lidocaine by syringe-to-syringe transfer. After lidocaine dissolution, 2 mg of epinephrine hydrochloride was added to the gel and mixed thoroughly. The gel was now a mixture Juvederm® Ultra Plus dermal filler with added lidocaine (0.3 wt %) and epinephrine (100 PPM). This mixture was filled into 1 mL Schott cyclic olefin copolymer syringes.

Dry forms of the gel mixture were prepared. To make the dry forms of the gel, the plungers, plunger heads, and caps of 10 of the syringes were removed and these syringes with gel were flash-frozen in liquid nitrogen. Those gels were then lyophilized at 0.02 Torr and −50° C. for 18 hrs. The gels formed a collapsed white cylinder of dried material in each syringe.

10 syringes containing wet forms of the gel (i.e. the gel mixtures which were not flash frozen in liquid nitrogen) and the 10 syringes containing the dry forms of the gel were then compared as follows.

The dry forms of the gel and wet forms of the gel were stored in the syringes until defined time-points.

The gels in dry form were rehydrated at the defined time point as follows. 5 mm septa were attached to the tips of the dry form syringes and 13 mm ID septum caps were attached to the luer lock outer regions. The plunger and plunger heads were replaced. Tape was used to expand the plunger to hold it in place, and the ends were sealed with paraffin film. A 27G needle attached to a running vacuum pump was inserted through the septa to draw a vacuum of 6 Torr for 5 minutes, The syringe was then detached from the vacuum, A 1 mL syringe of degassed water was equipped with a 27G needle which was inserted through the septa on the syringe of dried gel.

After the rehydration of the dry forms, the concentrations of epinephrine as well as adrenochrome (an oxidation product of epinephrine) within all of the gel samples that were stored in wet and dry forms were measured through extraction from the gel with acetonitrile and quantification by HPLC at the defined time-points and compared.

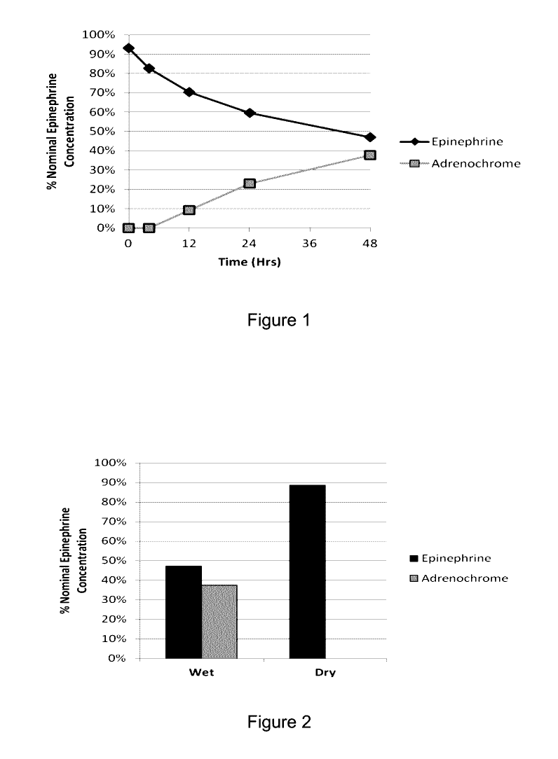

In wet form, more than 50% of the epinephrine had degraded within 48 hrs and the concentration of adrenochrome increased during this time (see FIG. 1). In dry form, epinephrine remained stable and was detected at levels near the extraction limit (see FIG. 2).

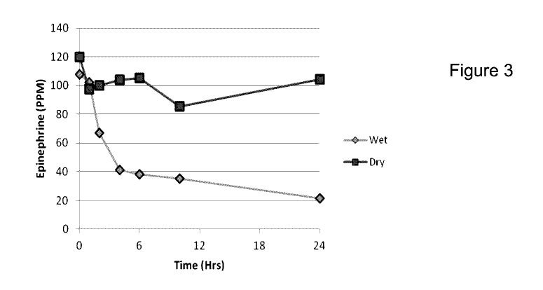

Accelerated aging of the gel mixtures (Juvederm® Ultra Plus containing 0.3% lidocaine and 100 PPM epinephrine) was conducted at 50° C. in both wet and dry forms. The presence of non-degraded epinephrine was determined at several time-points (FIG. 3). The epinephrine remained stable in the dry form of the gel, but degraded quickly when aged in the wet form of the gel.

### EXAMPLE 2

The plungers, plunger heads, and caps of 1 mL Schott cyclic olefin copolymer syringes containing 0.8 mL of Juvederm® 30 dermal filler were removed and the syringes with gel were frozen at −80° C. for 2 hrs. They were then lyophilized at 0.02 Torr and −50° C. for 18 hrs. After drying, 5 mm septa were attached to the tips and 13 mm ID septum caps were attached to the luer lock outer regions, The plunger and plunger heads were replaced. Tape was used to expand the plunger to hold it in place, and the ends were sealed with paraffin film. A 27G needle attached to a running vacuum pump was inserted through the septa to draw a vacuum of 6 Torr for 5 minutes. The syringe was then detached from the vacuum. A 1 mL syringe of degassed water was equipped with a 27G needle which was inserted through the septa on the syringe of dried Juvederm® 30. The gel was allowed to hydrate for 1 hr before rheological testing. The storage modulus (G′) at 2% strain and 5 Hz was 223 Pa after lyophilization and reconstitution compared to the original G′ of 144 Pa.

### EXAMPLE 3

Schott 1 mL cyclic olefin copolymer syringes without plungers, plunger heads, or caps were filled with 0.8 mL of four-arm epoxide crosslinked hyaluronic acid gels were flash-frozen in liquid nitrogen. They were then lyophilized at 0.02 Torr and −50° C. for 18 hrs, After drying, 5 mm septa were attached to the tips and 13 mm ID septum caps were attached to the leer lock outer regions. The plunger and plunger heads were replaced. Tape was used to expand the plunger to hold it in place, and the ends were sealed with paraffin film. A 27G needle attached to a running vacuum pump was inserted through the septa to draw a vacuum of 6 Torr for 5 minutes. The syringe was then detached from the vacuum. A 1 mL syringe of degassed water was equipped with a 27G needle which was inserted through the septa on the syringe of the dried gel. The gel was allowed to hydrate for 1 hr before rheological testing. The storage modulus (G′) at 2% strain and 5 Hz was 2260 Pa after lyophilization and reconstitution compared to the original G′ of 2260.

### EXAMPLE 4

A 33 year old woman informs her dermatologist that she is unhappy with the appearance of her face in that her nasolabial folds are deep and prominent, and cause her to look much older than her actual age. The physician recommends a dermal filler procedure which will add volume to the skin and reduce the appearance of the patient's nasolabial folds.

About an hour before the procedure is to take place, the dermatologist obtains a syringe containing a dermal filler composition comprising crosslinked hyaluronic acid, and a therapeutically effective amount of lidocaine and epinephrine, all in dry form, as described elsewhere herein. The composition appears as a collapsed white cylinder in the syringe barrel, and is under vacuum. The physician introduces about 2 mL of water into the syringe barrel using a syringe and needle inserted through a septum located at a distal end of the dermal filler syringe. The water contacts and rehydrates the dry dermal filler composition. The composition is allowed to rehydrate for 30 minutes to about one hour, The rehydrated gel now appears as a colorless gel, and comprises 24 mg/g of hyaluronic acid, 0.3% w/w lidocaine and 200 ppm of epinephrine.

The physician removes the septum from the syringe and attaches a 27 Gauge needle which is used to introduce the rehydrated gel into the patient's skin in the area of the nasolabial folds. The physician gently massages the treated areas to ensure even distribution of the gel in the skin. The physician applies ice to reduce the chance of bruising. Within an hour of treatment, the patient notices a favorable change in her appearance, as the nasolabial folds are less prominent and her face appears more youthful and fuller. The positive results last for about one year.

Although the invention has been described and illustrated with a certain degree of particularity, it is understood that the present disclosure has been made only by way of example, and that numerous changes in the combination and arrangement of parts, steps and elements can be resorted to by those skilled in the art without departing from the scope of the invention, as hereinafter claimed.

What is claimed is:

1. A method of making a material suitable for reconstitution and injection into skin as a dermal filler, the method comprising:
providing a gel comprising crosslinked hyaluronic acid and at least one additive;
lyophilizing the gel to obtain a dry gel suitable for reconstitution;
wherein the additive remains stable in the dry gel.

2. The method of claim 1 wherein the additive is lidocaine,

3. The method of claim 1 wherein the additive is epinephrine.

4. The method of claim 1 wherein the additive is phenylephrine.

5. The method of claim 1 wherein the at least one additive comprises lidocaine and epinephrine.

6. The method of claim 1 wherein the at least one additive comprises lidocaine and phenylephrine.

7. A method of making a dermal filler, the method comprising:
providing a dry gel comprising crosslinked hyaluronic acid and at least one additive; and
reconstituting the dry gel with an aqueous solution prior to injection into the skin.

8. The method of claim 7 wherein the step of reconstituting comprises adding soluble hyaluronic acid to the dry gel.
